(19) United States
(12) Patent Application Publication
(10) Pub. No.: US 2026/0216299 A1
(43) Pub. Date: Jul. 30, 2026

(54) Use of Cytoglobin in Preparation of Drug for Preventing and/or Treating Diabetes

(71) Applicant: Wenqi DONG, Guangzhou (CN)

(72) Inventors: Wenqi DONG, Guangzhou (CN); Zirong ZHANG, Guangzhou (CN)

(21) Appl. No.: 19/144,875

(22) PCT Filed: Sep. 26, 2023
(86) PCT No.: PCT/CN2023/121371

§ 371 Date: Aug. 7, 2025

(30) Foreign Application Priority Data
Dec. 30, 2022 (CN) 202211719582.X

(51) Int. Cl. A61K 38/42; A61P 1/18; A61P 3/06; A61P 3/10; A61P 5/50; A61P 13/12

(52) U.S. Cl. CPC A61K 38/42; A61P 1/18; A61P 3/06; A61P 3/10; A61P 5/50; A61P 13/12

(57) ABSTRACT

Use of cytoglobin in preparation of a drug for preventing and/or treating diabetes mellitus. The preventing and/or treating includes at least one selected from the group consisting of: (1) reducing a blood glucose level; (2) reducing a total cholesterol level; (3) reducing a triglyceride level; (4) reducing a low-density lipoprotein (LDL) level; (5) improving and restoring a normal morphological structure of a pancreatic tissue; (6) promoting proliferation of a pancreatic β-cell; (7) promoting insulin and glucagon secretion by pancreatic islets; and (8) improving renal steatosis and inflammatory cell infiltration.

### CROSS REFERENCE TO RELATED APPLICATION

This patent application is a national stage application of International Patent Application No. PCT/CN2023/121371, filed on Sep. 26, 2023, which claims the benefit and priority to Chinese Patent Application No. 202211719582.X, entitled “USE OF CYTOGLOBIN IN PREPARATION OF DRUG FOR PREVENTING AND/OR TREATING DIABETES”, filed with the China National Intellectual Property Administration (CNIPA) on Dec. 30, 2022, which is incorporated herein by reference in its entirety.

### TECHNICAL FIELD

The present disclosure belongs to the technical field of pharmaceutical formulations, in particular relates to use of cytoglobin in preparation of a drug for preventing and/or treating diabetes mellitus.

### BACKGROUND

Diabetes mellitus (DM) is a disease characterized by metabolic disorders leading to chronically elevated blood glucose levels in the body. Symptoms of hyperglycemia include frequent urination, increased thirst, and heightened hunger. If left untreated, DM can cause various complications. Acute complications include diabetic ketoacidosis, hyperosmolar hyperglycemic state, or death. Serious long-term complications encompass cardiovascular disease, stroke, chronic kidney disease, foot ulcers, and eye damage. Elevated blood glucose typically results from factors such as an unhealthy diet, lack of physical activity, impaired uptake and utilization of glucose by the body, and affected glycogen synthesis from glucose.

Type II diabetes (T2D) is the most common type of DM, occurring when the body cannot effectively produce or use insulin. Manifesting as relative insulin deficiency, this disease causes glucose to accumulate in the bloodstream. Over time, excessive blood glucose leads to severe problems: persistently elevated blood glucose, increased blood viscosity, local ischemia and hypoxia in multiple organs, abnormal glucose metabolism, and cellular damage from oxidative stress. Prolonged conditions result in damage to organs such as the eyes, kidneys, and nerves, potentially causing heart disease, stroke, and even limb necrosis. The etiology of type II diabetes is multifactorial, including genetic predisposition, dysfunction of downstream insulin receptors, and abnormalities in pancreatic B-cells. According to the latest data from the International Diabetes Federation (IDF), over 425 million people worldwide have been living with DM by 2017. This number is projected to rise to 629 million by 2040. Therefore, effective prevention and treatment of DM can significantly improve global quality of life, making the exploration of safe and effective novel therapies for DM an urgent task!

### SUMMARY

A purpose of the present disclosure is to provide use of cytoglobin in preparation of a drug for preventing and/or treating diabetes mellitus. The cytoglobin exhibits a significant therapeutic effect in treating diabetes mellitus.

The present disclosure provides use of cytoglobin in preparation of a drug for preventing and/or treating diabetes mellitus.

In some embodiments, the diabetes mellitus includes type II diabetes.

In some embodiments, the preventing and/or treating includes at least one selected from the group consisting of:
(1) reducing a blood glucose level;
(2) reducing a total cholesterol level;
(3) reducing a triglyceride level;
(4) reducing a low-density lipoprotein (LDL) level;
(5) improving and restoring a normal morphological structure of a pancreatic tissue;
(6) promoting proliferation of an pancreatic β-cell;
(7) promoting insulin and glucagon secretion by pancreatic islets; and
(8) improving renal steatosis and inflammatory cell infiltration.

In some embodiments, the cytoglobin includes recombinant human cytoglobin.

In some embodiments, an intake of the cytoglobin is 5 mg/kg.

In some embodiments, a method of the intake includes injection.

In some embodiments, the injection includes injecting a phosphate-buffered saline (PBS) solution of the cytoglobin.

Beneficial Effects: the present disclosure provides use of cytoglobin in preparation of a drug for preventing and/or treating diabetes mellitus. In the examples, cytoglobin is applied for the treatment of diabetes mellitus, particularly type II diabetes. Under conditions of persistent hyperglycemia in diabetes mellitus, elevated cytoglobin levels effectively clear oxidative damage caused by glucose metabolism byproducts in various tissues and organs throughout the body. This mitigates the onset and progression of DM-related complications, specifically manifested by: reducing blood glucose levels, lowering total cholesterol content, decreasing triglyceride levels, reducing LDL levels, improving and restoring the normal morphological structure of pancreatic tissue, promoting proliferation of pancreatic β-cells, enhancing insulin and glucagon secretion by pancreatic islets, and ameliorating renal steatosis and inflammatory cell infiltration. In conclusion, the cytoglobin described herein can be used to treat DM.

### BRIEF DESCRIPTION OF THE DRAWINGS

FIG. 1 shows the blood glucose changes in the modeling group versus the blank control group in C57BL/6J mice during model establishment; *, p<0.05; **, p<0.01; ***, p<0.001; ****, p<0.0001; the same applies below;

FIG. 2 shows the body weight changes in the modeling group versus the blank control group in C57BL/6J mice during model establishment;

FIG. 3 shows the pre-treatment blood glucose changes in randomly selected mice from the model group and the normal group;

FIG. 4 shows the pre-treatment body weight changes in randomly selected mice from the model group and the normal group;

FIG. 5 shows a comparison of the area under the curve (AUC) for the oral glucose tolerance test (OGTT) between the blank control group and the DM model group in mice;

FIG. 6 shows a comparison of blood glucose values among the placebo group, rhCygb treatment group, and EX-4 treatment group in mice; NS (no significant) indicates no significant difference;

FIG. 7 shows a comparison of blood glucose values among the blank group, placebo group, rhCygb treatment group, and EX-4 treatment group in mice; the p-values labeled in the figure represent the weekly comparison of blood glucose values between the PBS group and the rhCygb group;

FIG. 8 shows the results of the therapeutic effect of different treatment groups on total cholesterol in C57BL/6J mice, as detected by a T-CHO kit;

FIG. 9 shows the results of the therapeutic effect of different treatment groups on triglycerides in C57BL/6J mice, as detected by a TG kit;

FIG. 10 shows the results of the therapeutic effect of different treatment groups on high-density lipoprotein (HDL) in C57BL/6J mice, as detected by an HDL-C kit;

FIG. 11 shows the results of the therapeutic effect of different treatment groups on low-density lipoprotein (LDL) in C57BL/6J mice, as detected by an LDL-C kit;

FIGS. 12A-12D show H&E staining results of pancreatic tissue sections from mice in each group; FIGS. 12A, 12B, 12C and 12D represent the NC group, PBS group, rhCygb group, and EX-4 group, respectively;

FIG. 13 shows the statistical analysis results of the H&E staining of pancreatic tissue sections from mice in each group;

FIG. 14 shows renal steatosis and inflammatory cell infiltration in mice with DM before and after rhCygb treatment; hollow circle: atrophy of the renal capsule cavity; hollow quadrilateral: steatosis of renal tubular epithelial cells; hollow triangle: renal tubular dilation; hollow hexagon: inflammatory cell infiltration; hollow pentagon: renal tubular protein casts;

FIG. 15 shows the statistical analysis results of the PCNA-positive cell rate in different treatment groups;

FIGS. 16A-D show immunohistochemical staining results for PCNA (200× magnification);

FIGS. 16A, 16B, 16C and 16D represent the NC group, PBS group, rhCygb group, and EX-4 group, respectively;

FIG. 17 shows the statistical analysis results of the average optical density for Glucagon in different treatment groups;

FIG. 18 shows the statistical analysis results of the average optical density for Insulin in different treatment groups;

FIG. 19 show immunohistochemical staining results for Glucagon (200× magnification);

FIGS. 19A, 19B, 19C and 19D represent the NC group, PBS group, rhCygb group, and EX-4 group, respectively; and

FIG. 20 show immunohistochemical staining results for Insulin (200× magnification);

FIGS. 20A, 20B, 20C and 20D represent the NC group, PBS group, rhCygb group, and EX-4 group, respectively.

### DETAILED DESCRIPTION OF THE EMBODIMENTS

The present disclosure provides use of cytoglobin in preparation of a drug for preventing and/or treating diabetes mellitus.

In some embodiments of the present disclosure, the diabetes mellitus includes type II diabetes. The efficacy of rhCygb in treating DM is comprehensively evaluated by administering subcutaneous injections of rhCygb to C57BL/6J diabetic model mice established through long-term high-fat diet feeding. The therapeutic effects are specifically manifested as follows:
(1) reducing a blood glucose level;
(2) reducing a total cholesterol level;
(3) reducing a triglyceride level;
(4) reducing a low-density lipoprotein (LDL) level;
(5) improving and restoring a normal morphological structure of a pancreatic tissue;
(6) promoting proliferation of an pancreatic β-cell;
(7) promoting insulin and glucagon secretion by pancreatic islets; and
(8) improving renal steatosis and inflammatory cell infiltration.

In some embodiments of the present disclosure, the cytoglobin includes recombinant human cytoglobin (rhCygb). Cytoglobin (Cygb) is the fourth member of the globin superfamily in mammals, functioning as a hexacoordinate heme globin. Cygb has a molecular weight of 21.4 kDa, consists of 190 amino acids, and is located on chromosome segment 17q25.3. In the examples, the level of cytoglobin in the subject is increased by injection, with an injection dosage of 5 mg/kg. In some embodiments, the injection includes administering a PBS solution of the cytoglobin.

To further describe the present disclosure, the use of cytoglobin in preparation of a drug for preventing and/or treating diabetes mellitus provided by the present disclosure will be described below in detail with reference to drawings and examples, but they may not be construed as limiting the protection scope of the present disclosure.

In the examples of the present disclosure, unless otherwise specified, all materials and methods used are those routinely available to those skilled in the art.

### 1. Experimental Animals

50 three-week-old male C57BL/6J mice, specific pathogen-free (SPF) grade, with body weights of approximately 15-20 g, were purchased from Guangzhou Sebiona Biotech Co., Ltd.

### 2. Major Reagents and Instruments

TABLE 1
Reagents used in the examples

| | |
|---|---|
| D12492 formula high fat diet | Guangdong Medical Laboratory Animal |
| | Center |
| Exendin-4 | MedChemExpress, US |
| DMEM | Corning, US |
| Fetal bovine serum (FBS) | Corning, US |
| Trypsin | Sigma, US |
| 4% paraformaldehyde solution | ServiceBio |
| Dimethyl sulfoxide (DMSO) | Sigma, US |
| Rabbit anti-mouse Ki 67 primary antibody | Cell Signaling Technology, UK |
| Rabbit anti-mouse PCNA primary antibody | Cell Signaling Technology, UK |
| Rabbit anti-mouse Proglucagon (D16G10) XP ® | Cell Signaling Technology, UK |
| primary antibody | |
| Rabbit anti-mouse Insulin (C27C9) primary antibody | Cell Signaling Technology, UK |
| Guinea pig anti-mouse Insulin primary antibody | Abcam, US |
| Guinea pig anti-mouse PDX1 primary antibody | Abcam, US |
| Goat anti-rabbit secondary antibody | ZSGB-BIO, Beijing |
| Goat anti-guinea pig secondary antibody | ZSGB-BIO, Beijing |
| Bovine serum albumin (BSA) | Sigma, US |
| Triglyceride (TG) assay kit | Nanjing Jiancheng Bioengineering Institute |
| Total cholesterol (TC) assay kit | Nanjing Jiancheng Bioengineering Institute |
| High-density lipoprotein cholesterol (HDL-C) assay | Nanjing Jiancheng Bioengineering Institute |
| kit | |
| Low-density lipoprotein cholesterol (LDL-C) assay | Nanjing Jiancheng Bioengineering Institute |
| kit | |
| BrandFord protein concentration assay kit | Novoprotein Scientific Co., Ltd |

TABLE 2
Instruments used in the examples

| | |
|---|---|
| Protein gel imaging system | Bio-Rad, US |
| Protein purification system | Bio-Rad, US |
| Micro high-speed refrigerated centrifuge | Sigma, America |
| High-Speed Refrigerated Centrifuge | Thermo Fisher, US |
| UV spectrophotometer | Eppendorf, Germany |
| 650 microplate reader | Bio-Rad, US |
| Ultrasonic cell disruptor | Ningbo Scientz Biotechnology Co., Ltd. |
| Vertical electrophoresis apparatus | Bio-Rad, US |
| Paraffin slicer | Thermo Fisher, US |
| Vitality blood glucose meter | Roche, Switzerland |

### 3. Statistical Analysis

Statistical analysis is conducted using Prism 7/SPSS 13.0 software. All quantitative results are obtained from at least three independent repeated experiments. Measurement data are expressed as mean±standard error (mean±SE). Comparisons among multiple groups are analyzed by one-way analysis of variance (One-way ANOVA). Pairwise comparisons within groups are conducted using the LSD method if variances are homogeneous; otherwise, Dunnet's T3 test is applied. Comparisons between two samples are conducted using an independent-samples t-test. P<0.05 is considered statistically significant.

### Example 1

### 1. Modeling Method

50 SPF-grade male C57BL/6J mice were purchased from Guangzhou Sebiona Biotech Co., Ltd. Their health status and whether they were all male were checked. All male mice were divided into groups of 5 per cage, totaling ten cages, with cage numbering. Cages 1 to 8 were designated as the high-fat diet modeling group and fed a high-fat diet with ad libitum feeding and drinking. Cages 9 and 10 were designated as the normal diet modeling group and fed standard chow with ad libitum feeding and drinking. All mice were marked with tail lines for identification. During modeling, body weight and fasting blood glucose (FBG) levels of all mice were measured weekly, and tail markings were reinforced. The blood glucose measurement procedure involved removing all feed from cage lids at 9:00 PM every Thursday and replacing bedding to ensure no food residue remained; mice retained free access to water. At 9:00 AM every Friday, body weight was measured, the tail tip was clipped, and approximately 1-2 μL of (a drop of) blood was applied to a Roche Accu-Chek® Active test strip on a glucometer to determine FBG. Monitoring of blood glucose and body weight continued until a statistically significant difference in FBG emerged between the high-fat diet modeling group and the normal diet modeling group, with a total modeling duration of 58 weeks. Mice were maintained under SPF conditions at a room temperature of 22° C. with a 12-h light cycle.

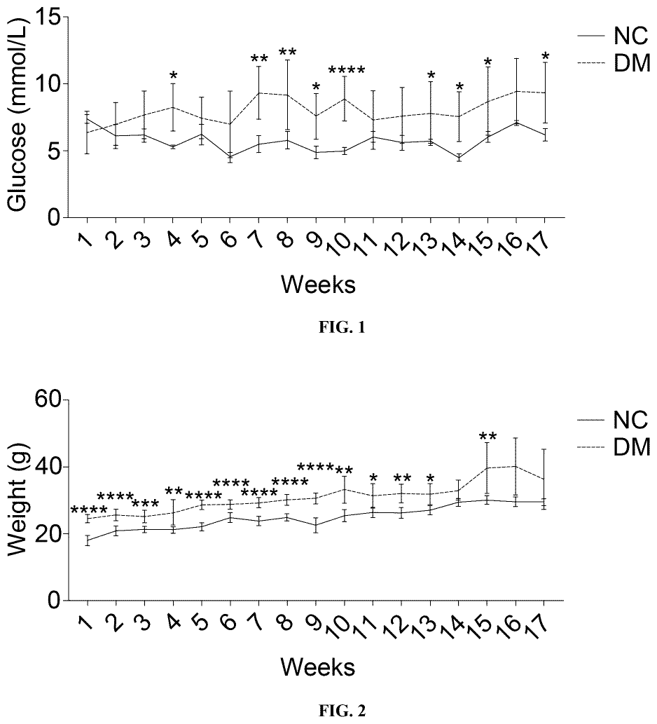

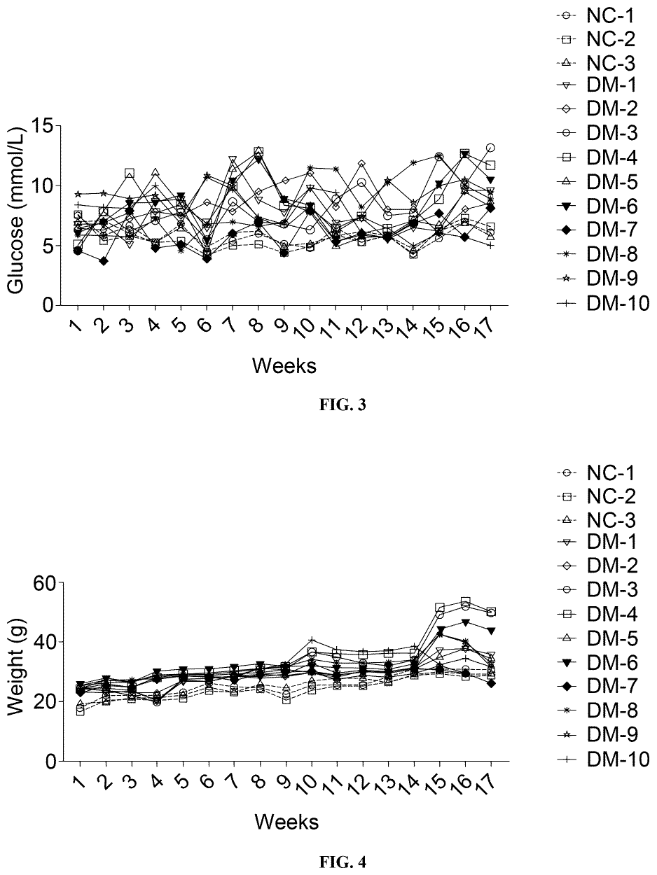

As shown in FIG. 1 to FIG. 4, a statistically significant difference in blood glucose levels between the NC group and the modeling group began to appear from the 4th week of modeling, but the changes were unstable. During the subsequent fifth and sixth weeks, no significant difference was observed between the NC group and the C57BL/6J modeling group. From the seventh week onward, the difference in blood glucose levels between the NC group and the C57BL/6J modeling group stabilized consistently, indicating the significance of the high-fat diet-induced DM modeling approach. This model required prolonged feeding, with results tending to stabilize over time. However, in later stages when mice developed complications of DM such as skin diseases and eye disorders, the blood glucose levels in the C57BL/6J modeling group began to show a downward trend (FIG. 1). Body weight statistics revealed that after high-fat diet feeding, the modeling group exhibited significantly higher body weights than the normal diet group, with a stable and pronounced difference. After prolonged high-fat diet feeding, substantial weight fluctuations occurred within the DM model group due to varying severity of complications among individual mice. Nevertheless, mice without complications maintained very high body weights (FIG. 2). Blood glucose levels in C57BL/6J mice fed solely a high-fat diet showed considerable volatility, unlike the sustained hyperglycemia seen in models with chemical-induced pancreatic β-cell damage or genetic defects. However, the overall trend of red section (diabetes mellitus modeling group, DM modeling group) displayed a clear upward trajectory compared to the black section (normal control group) (FIG. 3). Body weights in the high-fat diet-fed C57BL/6J mice were consistently and significantly higher than those in the normal diet modeling group, with maximum weights exceeding 55 g. Notably, as modeling duration extended, some mouse individuals in the DM C57BL/6J group began exhibiting symptoms suggestive of severe DM complications (e.g., non-healing skin lesions, vitreous opacity, cataracts, stroke-induced paralysis). Consequently, the body weight of these affected mouse individuals declined sharply (FIG. 4).

### 2. Grouping and Treatment

1) After successful 58-week modeling, 40 mice from the modeling group underwent an OGTT to confirm statistically significant glucose intolerance decrease. Statistical analysis verified a significant difference in FBG between the modeling group and the normal diet group.

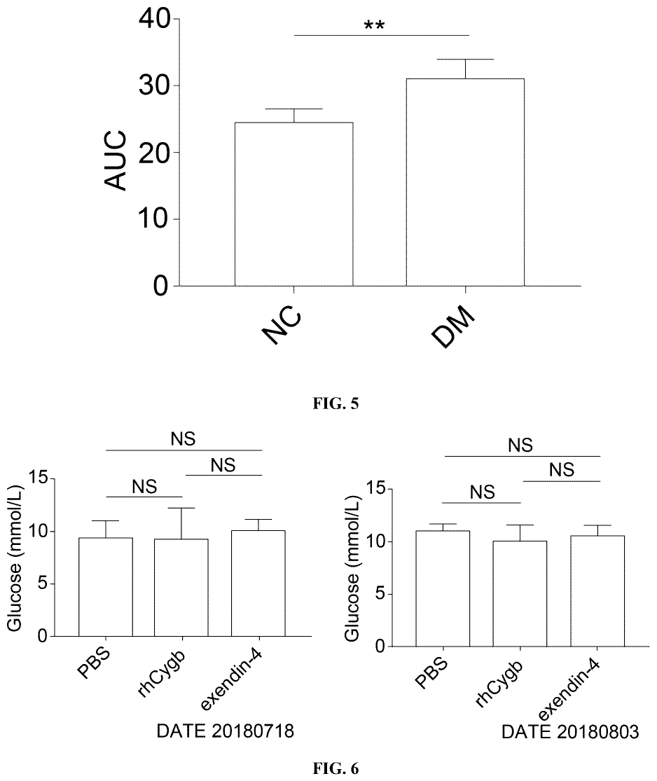

The AUC for blood glucose changes within two hours post-glucose gavage was shown in FIG. 5, confirming impaired glucose tolerance in the DM group.
2) Successfully modeled mice were divided into three groups based on no statistical difference in blood glucose within groups and statistical difference in blood glucose between groups: wherein the three groups are rhCygb treatment group (5 mg/kg subcutaneous injection), exendin-4 treatment group (50 μg/kg subcutaneous injection), and placebo group (0.1 mL PBS subcutaneous injection) (grouping without re-caging, mice remained in original cages), respectively.

Given that type II diabetes modeled by high-fat diet feeding mirrors natural pathogenesis with individual variability, the significant differences in blood glucose between different mice could not be addressed through random grouping for the experiment. Statistical grouping was employed, with the results shown in FIG. 6. FBG levels showed no significant difference between placebo group, rhCygb treatment group, and EX-4 treatment group over two consecutive weeks, validating their use for subsequent experiments.

In summary, compared to normal controls: model group mice developing severe DM complications partly exhibited sharp weight decline as non-healing skin lesions, vitreous opacity, cataracts, stroke-induced paralysis appeared. And compared to normal controls, model group mice demonstrated impaired glucose tolerance. A DM model in C57BL/6J mice was successfully established, and the grouping was reasonable.

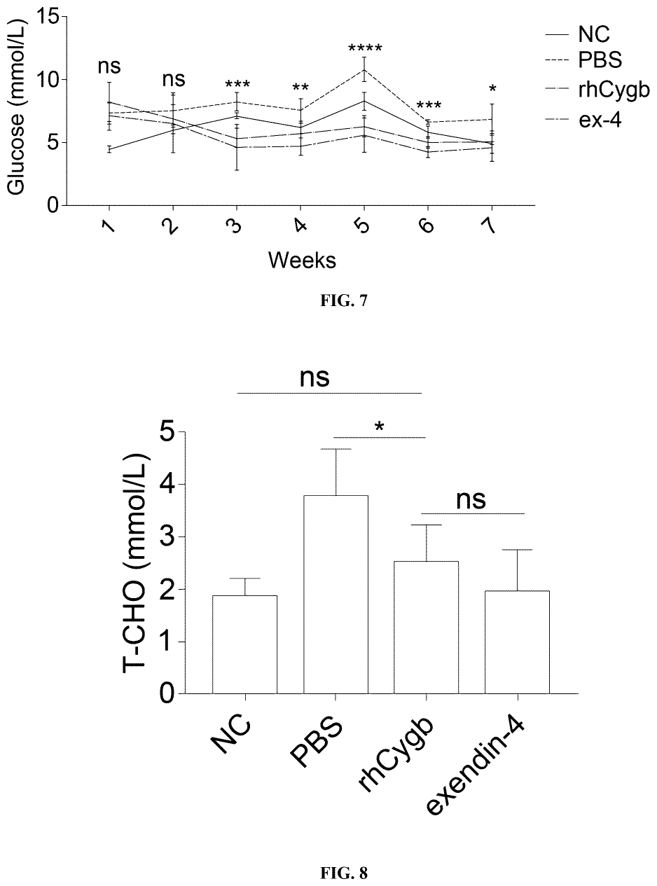

The mice of placebo group, rhCygb treatment group, and EX-4 treatment group received daily subcutaneous injections of PBS, rhCygb, or EX-4, respectively for 2 months (frequency adjusted based on survival status of mice). Weekly blood glucose and body weight monitoring of mice revealed that rhCygb treatment significantly reduced blood glucose after 6 weeks, with a stable efficacy similar to that of the EX-4 group (FIG. 7).
3) Throughout the experiment, C57BL/6J mice were monitored for: fur condition, food intake, fecal abnormalities, limb mobility, mortality events, and causes of death.
4) After 28 d of treatment: blood was collected from eyeballs for detection of four blood lipid indicators. Mice were immediately sacrificed by cervical dislocation after blood collection and dissected for tissue collection. Liver tissue, pancreas, kidneys, heart, lungs, stomach, and spleen were sequentially collected for biochemical and pathological examination as detailed below.
   3. Measurement of four blood lipid items
1) Total Cholesterol (T-CHO) Measurement: prepared serum was diluted tenfold with physiological saline at a 1:9 ratio. Into each well of a 96-well plate, 2.5 μL of deionized water, 2.5 μL of standard, and 2.5 μL of sample were sequentially added. Then, 250 μL of working solution was added to each well, thoroughly mixed, and incubated at 37° C. for 10 min in an electric thermostatic incubator. Absorbance at 510 nm was measured using a microplate reader. Cholesterol content was calculated according to the formula:

$$Total cholesterol ( mmol / L ) = ( Sample OD - Blank OD ) / ( Standard OD - Blank OD ) \times Standard concentration \times 10$$

Serum from each treated C57BL/6J mouse was assayed using a total cholesterol assay kit. As shown in FIG. 8, the rhCygb treatment group and exendin-4 treatment group after treatment exhibited a statistically significant reduction in total cholesterol levels compared to the placebo group. Furthermore, no significant difference was observed between the treated groups (rhCygb treatment group and exendin-4 treatment group) and the blank control group, indicating effective therapeutic outcomes.
2) Triglyceride (TG) Measurement: prepared serum was diluted tenfold with physiological saline at a 1:9 ratio. Into each well of a 96-well plate, 2.5 μL of deionized water, 2.5 μL of standard, and 2.5 μL of sample were sequentially added. Then, 250 μL of working solution was added to each well, thoroughly mixed, and incubated at 37° C. for 10 min in an electric thermostatic incubator. Absorbance at 510 nm was measured using a microplate reader. Triglyceride content was calculated according to the formula:

$$Triglyceride ( mmol / L ) = ( Sample OD - Blank OD ) / ( Standard OD - Blank OD ) \times Standard concentration \times 10$$

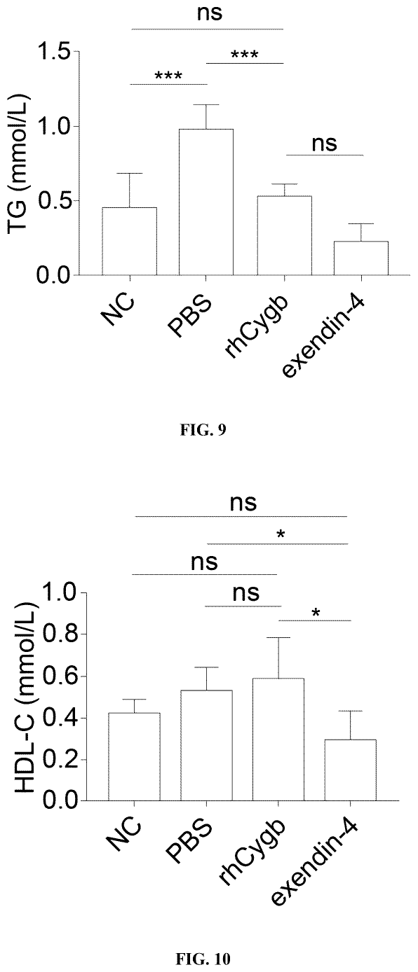

Serum from each treated C57BL/6J mouse was assayed using a triglyceride assay kit. As shown in FIG. 9, no significant difference was observed in triglyceride levels between the rhCygb treatment group and either the exendin-4 treatment group or the NC group. However, the rhCygb group exhibited a statistically significant reduction in triglyceride levels compared to the placebo group, demonstrating effective therapeutic efficacy.
3) High-Density Lipoprotein Cholesterol (HDL-C) Measurement: prepared serum was diluted tenfold with physiological saline at a 1:9 ratio. Into each well of a 96-well plate, 2.5 μL of deionized water, 2.5 μL of standard, and 2.5 μL of sample were sequentially added. Then, 180 μL of working solution R1 was added to each well, thoroughly mixed, and incubated at 37° C. for 5 min in an electric thermostatic incubator. Absorbance at 546 nm (A1) was measured using a microplate reader. Subsequently, 60 μL of working solution R2 was added to each well, thoroughly mixed, and incubated at 37° C. for 5 min. Absorbance at 546 nm (A2) was measured again. HDL-C content was calculated according to the formula:

$$HDL - C ( mmol / L ) = ( ( Sample A 2 - Sample A 1 ) - ( Blank A 2 - Blank A 1 ) ) / ( ( Standard A 2 - Standard A 1 ) - ( Blank A 2 - Blank A 1 ) ) \times Standard concentration \times 10$$

Serum from each treated C57BL/6J mouse was assayed using an HDL-C assay kit. As shown in FIG. 10, no statistically significant differences were observed in HDL-C levels between any treatment groups and the NC group. Although the rhCygb group exhibited numerically higher HDL-C levels on average compared to other groups, the results were insufficient to demonstrate statistically that rhCygb treatment could elevate HDL-C levels in DM mice.
4) Low-Density Lipoprotein Cholesterol (LDL-C) Measurement: prepared serum was diluted tenfold with physiological saline at a 1:9 ratio. Into each well of a 96-well plate, 2.5 μL of deionized water, 2.5 μL of standard, and 2.5 μL of sample were sequentially added. Then, 180 μL of working solution R1 was added to each well, thoroughly mixed, and incubated at 37° C. for 5 min in an electric thermostatic incubator. Absorbance at 546 nm (A1) was measured using a microplate reader. Subsequently, 60 μL of working solution R2 was added to each well, thoroughly mixed, and incubated at 37° C. for 5 min in an electric thermostatic incubator. Absorbance at 546 nm (A2) was measured again. LDL-C content was calculated according to the formula:

$$LDL - C ( mmol / L ) = ( ( Sample A 2 - Sample A 1 ) - ( Blank A 2 - Blank A 1 ) ) / ( ( Standard A 2 - Standard A 1 ) - ( Blank A 2 - Blank A 1 ) ) \times Standard concentration \times 10$$

Serum from each treated C57BL/6J mouse was assayed using an LDL-C assay kit. As shown in FIG. 11, the rhCygb treatment group and exendin-4 treatment group after treatment exhibited a statistically significant reduction in LDL-C levels compared to the placebo group. Furthermore, no significant difference was observed between the treated groups (rhCygb treatment group and exendin-4 treatment group) and the blank control group, confirming effective therapeutic efficacy.

Collectively, rhCygb treatment for 2 months significantly reduced blood glucose in mice, with stable efficacy similar to that of the EX-4 group. Moreover, rhCygb treatment effectively lowered total cholesterol, triglycerides, and LDL-C levels in the blood.

### 4. H&E Staining

Tissue paraffin blocks were sectioned. Paraffin was dissolved using xylene solution, followed by sequential immersion and washing in absolute ethanol, 90% alcohol, 70% alcohol, 50% alcohol, 25% alcohol, and distilled water. Sections washed in distilled water were stained in hematoxylin aqueous solution for several minutes, differentiated briefly in acid water and ammonia water, each lasting a few seconds, then rinsed under running water for 1 h before immersion in distilled water. Sections were removed from distilled water and dehydrated sequentially in 70% and 90% alcohol (10 min each). Dehydrated sections were immersed in alcoholic eosin solution for staining, lasting 2-3 min. The stained sections were immersed in absolute ethanol for dehydration. Sections were washed in xylene solution until they became transparent. Transparent sections were mounted by dripping gum and covering with coverslips. After approximately 30 min, labels were affixed to the slides for marking. Observation and description were performed under an optical microscope (Microscope: NIKON Eclipse ci; imaging system: NIKON Digital Sight DS-FI2, MADE IN JAPAN). Images were captured at 200× and 400× magnification.

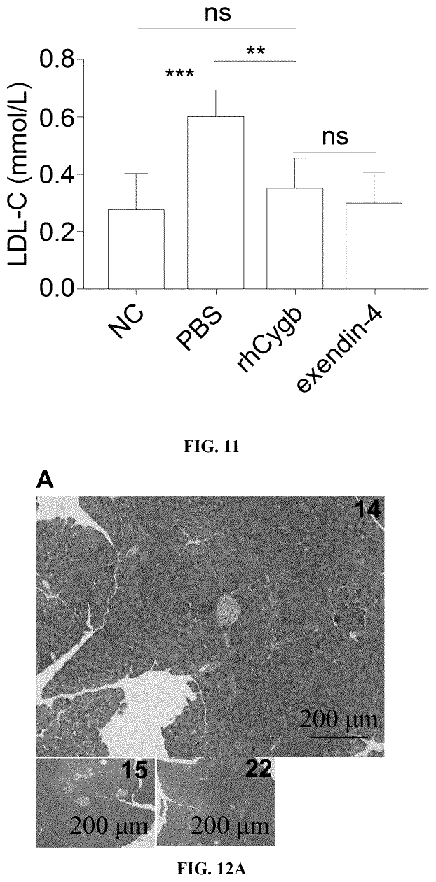

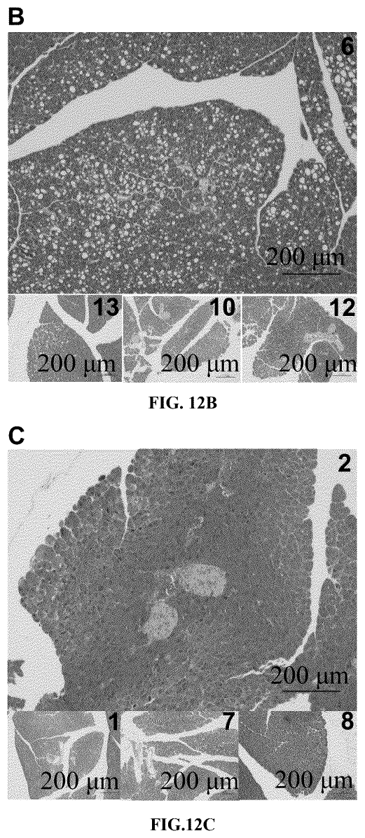

As shown in FIGS. 12A-12D, pancreatic tissue from the PBS group exhibited extensive acinar cell steatosis with variably sized circular vacuoles in the cytoplasm, suggesting vacuolar degeneration of β-cells. In contrast, the rhCygb treatment group displayed normal pancreatic islet morphology without significant inflammation. Minimal acinar cell degeneration was observed, comparable to the NC group, indicating that rhCygb treatment effectively improved and restored normal pancreatic tissue. Conversely, the EX-4 group showed persistent acinar steatosis similar to the PBS group, demonstrating no restorative effect from EX-4 on pancreatic steatosis.

The severity of lesions observed in the HE-stained pathological sections of each mouse was graded. This ordinal data was analyzed using the K Independent Samples non-parametric test in SPSS software, and the result indicated a statistically significant difference between groups. As SPSS lacks pairwise comparison for multiple independent samples, all data were ranked and converted into quantitative data, then subjected to one-way ANOVA. Results (FIG. 13) confirmed no significant difference between rhCygb and NC groups, while significant differences existed between PBS vs. NC and PBS vs. rhCygb, demonstrating rhCygb's efficacy in ameliorating pancreatic steatosis.

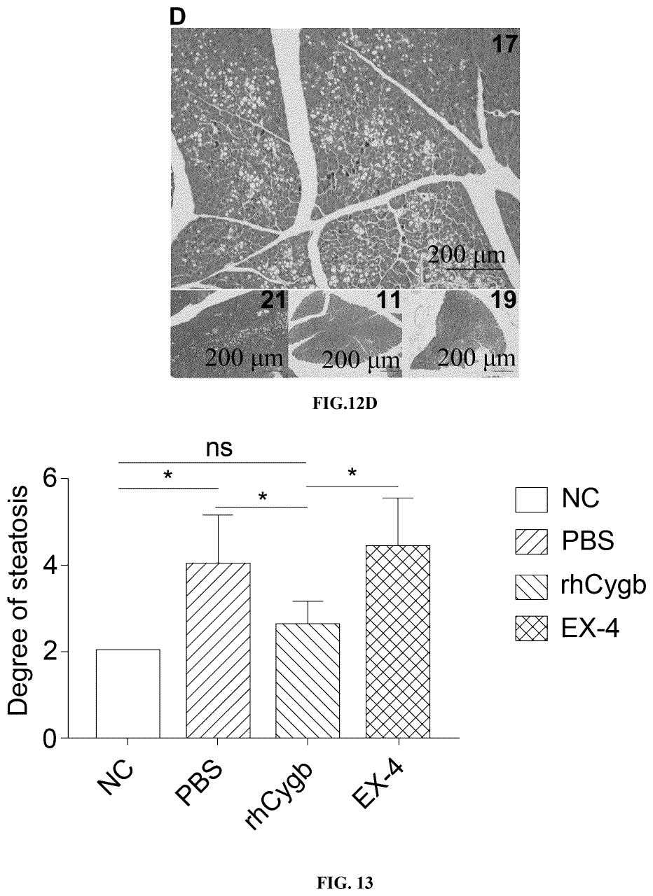

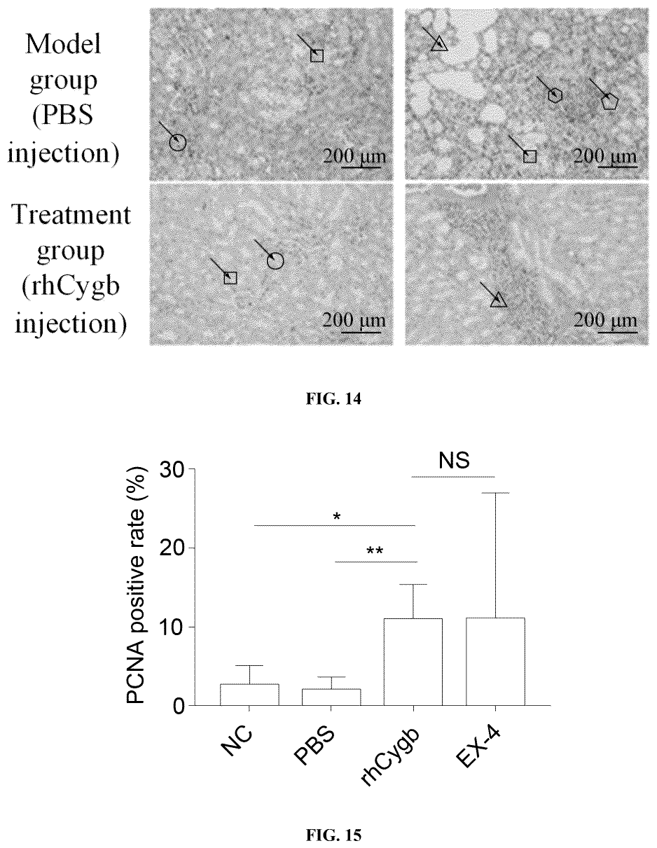

FIG. 14 revealed marked renal steatosis and inflammatory cell infiltration in the PBS group (model group). The rhCygb treatment group exhibited near-normal renal morphology without significant inflammatory cells. This confirmed rhCygb showing an ability to improve and restore normal structural morphology of renal tissue.

### 5. Immunohistochemistry

Pancreatic tissues were sectioned at 4 μm and baked at 60° C. for 2 h until paraffin melted. Sections underwent hematoxylin-eosin (H&E) staining.

Sections were immersed in methanol containing 3% hydrogen peroxide (endogenous peroxidase blocker) for 15 min. After PBS (10 mM, pH=7.0) rinsing, sections were incubated at 25° C. for 1 h with antibody cocktails (rabbit anti-glucagon monoclonal antibody: anti-somatostatin antibody=1:1), or separately with: anti-mouse insulin antigen monoclonal antibody, anti-proliferating cell nuclear antigen (PCNA) monoclonal antibody, rabbit anti-Ki67 monoclonal antibody. Alternatively, sections were incubated with guinea pig anti-PDX-1 monoclonal antibody at 4° C. for 14 h. Nuclei were counterstained blue with hematoxylin and sections were subjected to morphometric analysis.

PCNA Analysis method: The immunohistochemistry results were analyzed for percentage using Image-Pro Plus 6.0 (Media Cybernetics, Rockville, MD, USA), at least 3 random field of view at 200× per section within a group were captured. When taking pictures, the tissue should try to fill the entire field of view, ensuring consistent background lighting in each photo. Image-Pro Plus 6.0 software was used to select the same brown-yellow cell nucleus as the uniform standard for determining positive cells in all photos, while the same blue cell nucleus was selected as the total cell. Each photo was analyzed to obtain the number of positive cells and the total number of cells in each photo. Then, the percentage of positive cells was calculated, as: Positive cell percentage (%)=(Positive nuclei count/Total nuclei count)*100 was calculated as the positive rate.

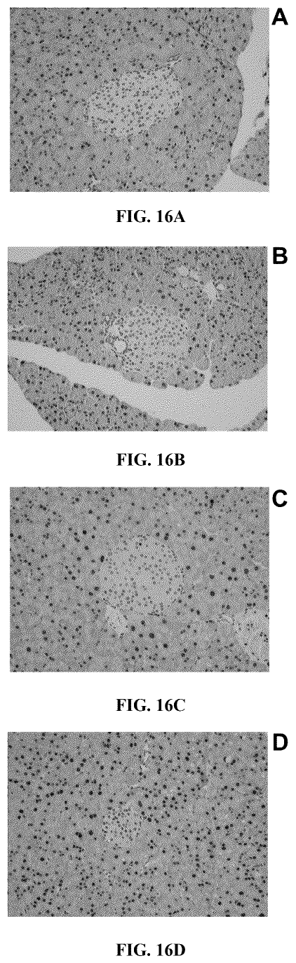

Positive staining for PCNA, a marker of cell proliferation, was analyzed for cell positive rate using Image-Pro Plus 6.0 software. Statistical analysis revealed no significant difference in PCNA-positive cell rates between the rhCygb treatment group and EX-4 treatment group, while significant differences existed between rhCygb treatment group vs. PBS group and rhCygb treatment group vs. NC group (FIG. 15 to FIG. 16D). These results indicated that 4-week rhCygb treatment significantly enhanced PCNA staining intensity in C57BL/6J mice, achieving efficacy comparable to the positive drug treatment. This demonstrated that rhCygb treatment could promote pancreatic β-cell proliferation.

Glucagon/Insulin Analysis Method: The immunohistochemistry results were analyzed for average optical density analysis using Image-Pro Plus 6.0 (Media Cybernetics, Inc., Rockville, MD, USA). For each section with a group, at least 3 fields of view at 200× magnification were captured. When taking pictures, the tissue should try to fill the entire field of view, ensuring consistent background lighting in each photo. Image-Pro Plus 6.0 software was used to select the same brown-yellow color on the islets as the unified standard for judging positivity in all photos. The integrated optical density (IOD) value of positive staining on the pancreatic islet and the pixel area (AREA) of the pancreatic islet were analyzed for each photo. The average optical density value (AO value) was calculated as AO=IOD/AREA. A higher AO value indicates a higher level of positive expression.

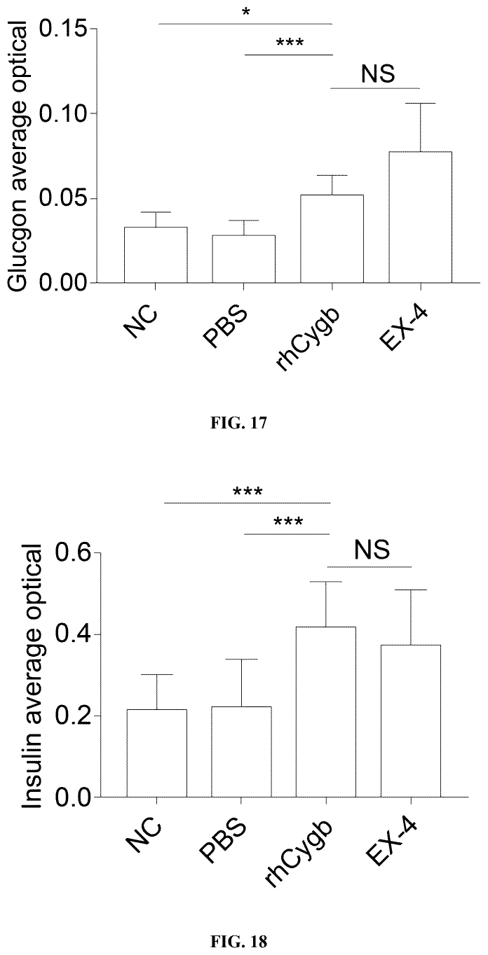

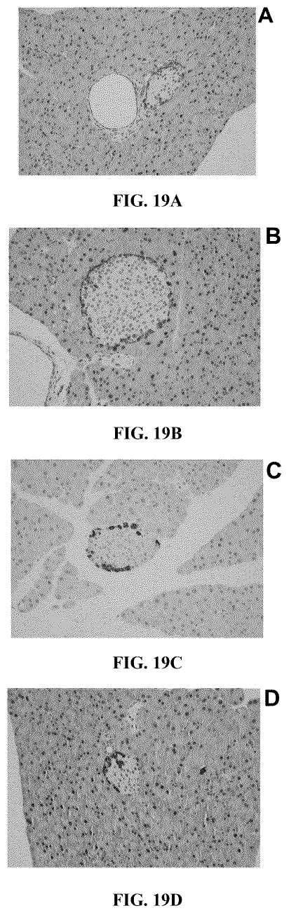

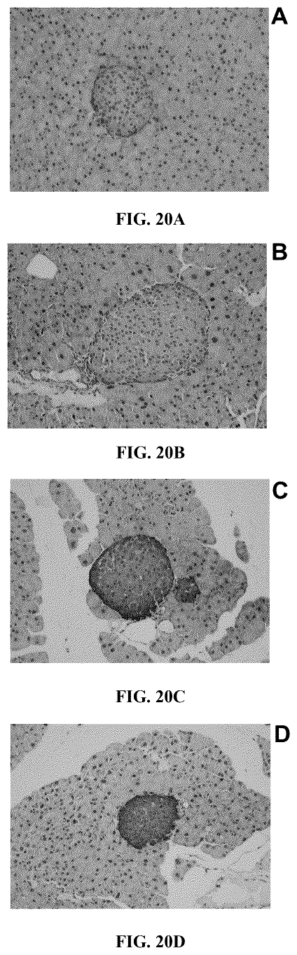

As shown in FIG. 17 to FIG. 20D, analysis of cell positive rate via Image-Pro Plus 6.0 revealed through statistical analysis that the expression levels of both insulin and glucagon in the rhCygb treatment group showed no statistically significant difference compared to the EX-4 groups. Furthermore, the expression levels of insulin and glucagon in the rhCygb treatment group showed statistically significant differences compared to both the PBS group and the NC group. This confirmed rhCygb treatment in promoting insulin and glucagon secretion in the pancreatic islets.

In conclusion, rhCygb exerts glucose-lowering effects by promoting pancreatic islet cell proliferation and enhancing insulin expression. These findings demonstrate that the cytoglobin of the present disclosure is applicable for DM treatment.

Although the above examples have described the present disclosure in detail, they are only a part of, not all of, the examples of the present disclosure. Other examples may also be obtained by persons based on the examples without creative efforts, and all of these examples shall fall within the protection scope of the present disclosure.

1. A method for preparing a drug for preventing and/or treating diabetes mellitus, comprising adding cytoglobin to the drug.

2. The method according to claim 1, wherein the diabetes mellitus comprises type II diabetes.

3. The method according to claim 1, wherein the preventing and/or treating comprises at least one selected from the group consisting of:
(1) reducing a blood glucose level;
(2) reducing a total cholesterol level;
(3) reducing a triglyceride level;
(4) reducing a low-density lipoprotein (LDL) level;
(5) improving and restoring a normal morphological structure of a pancreatic tissue;
(6) promoting proliferation of a pancreatic β-cell;
(7) promoting insulin and glucagon secretion by pancreatic islets; and
(8) improving renal steatosis and inflammatory cell infiltration.

4. The method according to claim 1, wherein the cytoglobin comprises recombinant human cytoglobin.

5. The method according to claim 1, wherein an intake dose of the cytoglobin is 5 mg/kg.

6. The method according to claim 5, wherein a method of intake comprises injection of a phosphate-buffered saline (PBS) solution of the cytoglobin.

7. A method for preventing and/or treating diabetes mellitus using cytoglobin, comprising increasing a content of the cytoglobin in a subject.

8. The method according to claim 7, wherein the content of the cytoglobin in the subject is increased by injection.

9. The method according to claim 8, wherein a dosage of the injection is about 5 mg/kg.

10. The method according to claim 8, wherein the injection comprises injecting a PBS solution of the cytoglobin.

11. A drug for preventing and/or treating diabetes mellitus, comprising cytoglobin.

12. The method according to claim 5, wherein the cytoglobin comprises recombinant human cytoglobin.
